(19) United States
(12) Patent Application Publication
(10) Pub. No.: US 2015/0224320 A1
(43) Pub. Date: Aug. 13, 2015

(54) MULTI-CHAMBER LEADLESS PACEMAKER SYSTEM WITH INTER-DEVICE COMMUNICATION

(71) Applicant: Cardiac Pacemakers, Inc., St. Paul, MN (US)

(72) Inventor: Jeffrey E. Stahmann, Ramsey, MN (US)

(21) Appl. No.: 14/618,396

(22) Filed: Feb. 10, 2015

Related U.S. Application Data
(60) Provisional application No. 61/938,020, filed on Feb. 10, 2014.

(51) Int. Cl. A61N 1/365; A61N 1/05

(52) U.S. Cl. CPC A61N 1/36592; A61N 1/0587

(57) ABSTRACT

Systems and methods for communicating cardiac events between a plurality of implantable medical devices. In one example, a system comprises a first leadless cardiac pacemaker (LCP) implantable at a first heart site and a second leadless cardiac pacemaker (LCP) implantable at a second heart site. The first LCP is configured to communicate information related to a cardiac event that is sensed by the first LCP at the first heart site to the second LCP, and the second LCP is configured to deliver one or more cardiac pacing pulses to one or more pacing electrodes of the second LCP based, at least in part, on the communicated information received from the first LCP.

### CROSS-REFERENCE TO RELATED APPLICATIONS

This application claims priority under 35 U.S.C. §119 to U.S. Provisional Application Ser. No. 61/938,020, filed Feb. 10, 2014, the entirety of which is incorporated herein by reference.

### TECHNICAL FIELD

The present disclosure generally relates to pacemakers, and more particularly, to systems and methods for coordinating detection and/or treatment of abnormal heart activity using multiple implanted devices within a patient.

### BACKGROUND

Pacemakers can be used to treat patients suffering from various heart conditions that can result in a reduced ability of the heart to deliver sufficient amounts of blood to a patient's body. In some cases, heart conditions may lead to rapid, irregular, and/or inefficient heart contractions. To help alleviate some of these conditions, various devices (e.g., pacemakers, defibrillators, etc.) can be implanted in a patient's body. Such devices are often used to monitor heart activity and provide electrical stimulation to the heart to help the heart operate in a more normal, efficient and/or safe manner.

### SUMMARY

The present disclosure relates generally to systems and methods for coordinating detection and/or treatment of abnormal heart activity using multiple implanted devices within a patient. In some cases, the devices may be implanted within separate chambers of the heart and may communicate information between the various chambers for improving detection and treatment of cardiac rhythm abnormalities. It is contemplated that the multiple implanted devices may include, for example, pacemakers with leads, leadless pacemakers, defibrillators, sensors, neuro-stimulators, and/or any other suitable implantable devices, as desired.

The above summary is not intended to describe each embodiment or every implementation of the present disclosure. Advantages and attainments, together with a more complete understanding of the disclosure, will become apparent and appreciated by referring to the following description and claims taken in conjunction with the accompanying drawings.

### BRIEF DESCRIPTION OF THE DRAWINGS

The disclosure may be more completely understood in consideration of the following description of various illustrative embodiments in connection with the accompanying drawings, in which:

FIG. 1 illustrates a block diagram of an exemplary medical device that may be used in accordance with various examples of the present disclosure;

FIG. 2 illustrates an exemplary leadless cardiac pacemaker (LCP) having electrodes, according to one example of the present disclosure;

FIG. 3 is a schematic diagram of an exemplary medical system that includes multiple leadless cardiac pacemakers (LCPs) and/or other devices in communication with one another example of the present disclosure;

FIG. 4 is a schematic diagram of the a system including an LCP and another medical device, in accordance with another example of the present disclosure;

FIG. 5 is a schematic diagram illustrating a multiple leadless cardiac pacemaker (LCP) system in accordance with another example of the present disclosure;

FIG. 6 is a schematic diagram illustrating a multiple leadless cardiac pacemaker (LCP) system, in accordance with yet another example of the present disclosure;

FIG. 7 is a graphical depiction of sensed and paced cardiac events showing an illustrative method of multi-chamber therapy, in accordance with the present disclosure;

FIG. 8a is a graphical depiction of sensed and paced cardiac events including communication signals, in accordance with the present disclosure;

FIG. 8b is a graphical depiction of an illustrative communication signal, in accordance with the present disclosure;

FIG. 8c is a graphical depiction of another illustrative communication signal, in accordance with the present disclosure;

FIG. 8d is a graphical depiction of yet another illustrative communication signal, in accordance with the present disclosure;

FIG. 9 is a flow diagram of an illustrative method that may be implemented by a medical device system, such as those medical device systems described with respect to FIGS. 3-6; and

FIG. 10 is a flow diagram of an illustrative method that may be implemented by a medical device system, such as those medical device systems described with respect to FIGS. 3-6.

While the disclosure is amenable to various modifications and alternative forms, specifics thereof have been shown by way of example in the drawings and will be described in detail. It should be understood, however, that the intention is not to limit aspects of the disclosure to the particular illustrative embodiments described. On the contrary, the intention is to cover all modifications, equivalents, and alternatives falling within the spirit and scope of the disclosure.

### DESCRIPTION

The following description should be read with reference to the drawings in which similar elements in different drawings are numbered the same. The description and the drawings, which are not necessarily to scale, depict illustrative embodiments and are not intended to limit the scope of the disclosure.

Normal, healthy hearts operate by coordinating contraction of the atria and the ventricles. For example, the atria of a heart normally contract first, thereby forcing blood into corresponding ventricles. Only after the blood has been pumped into the ventricles do the ventricles contract, forcing the blood into the arteries and throughout the body. Various conditions may cause such coordinated contraction to become un-synchronized in a patient. Synchronized contraction across the multiple chambers of the heart can help to increase the pumping capacity of the heart. In some cases, the atria may start to beat too fast, and sometimes fibrillate. During these periods, it may be desirable to not synchronize the ventricle with the atrium and pace the ventricles independent of the atrium.

In order to assist patients who experience constant or intermittent un-synchronized contractions, various medical devices may be used to sense when uncoordinated contractions occur and to deliver electrical pacing therapy to the various chambers of the heart in order to coordinate the contractions. For example, medical device systems may be used to sense generated or conducted cardiac electrical signals that are indicative of a chamber contraction. In some cases, such medical device systems may be used to detect such signals in different chambers of the heart in order to distinguish between, for example, atrial and ventricular contractions. In some cases, such systems may deliver electrical stimulation, for example pacing pulses, to help the chambers contract in a more synchronous manner.

Multi-device systems can introduce unique challenges for implementing such multi-chamber therapy. In multi-device systems, two separate devices may be responsible for sensing cardiac events in different chambers and delivering electrical stimulation to the different chambers. In some instances, each of the devices may be able to detect and/or deliver electrical stimulation to one chamber of the heart. The multiple devices of such systems may be configured to communicate sensed cardiac events and other information to the other devices in order to safely and effectively deliver electrical stimulation to the various chambers. The present disclosure describes various techniques for communicating cardiac events between the various devices of such multi-device systems.

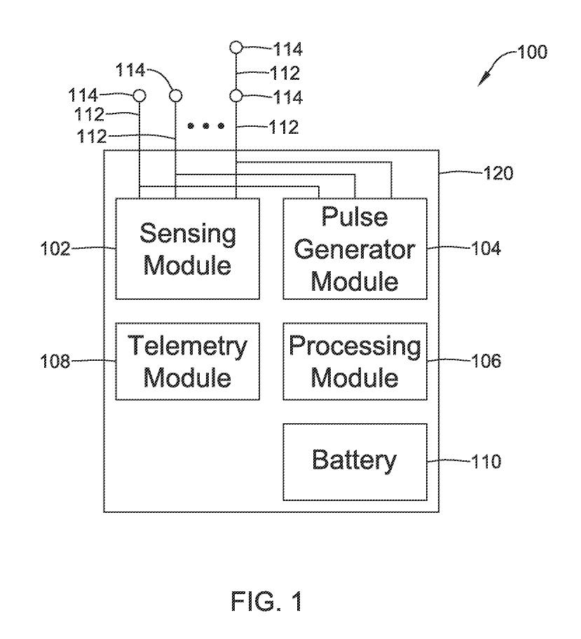

FIG. 1 illustrates a block diagram of an exemplary medical device 100 (referred to hereinafter as, MD 100) that may be used in accordance with various examples of the present disclosure. In some cases, the MD 100 may be used for sensing cardiac events, determining occurrences of arrhythmias, and delivering electrical stimulation. In some instances, MD 100 can be implanted within a patient's body, at a particular location (e.g., in close proximity to the patient's heart), to sense and/or regulate the cardiac events of the heart. In other examples, MD 100 may be located externally to a patient to sense and/or regulate the cardiac events of the heart. Cardiac contractions generally result from electrical signals that are intrinsically generated by a heart, but may also result from electrical stimulation therapy delivered by medical devices, such as MD 100. These electrical signals conduct through the heart tissue, causing the muscle cells of the heart to contract. MD 100 may include features that allow MD 100 to sense such generated or conducted cardiac electrical signals, or cardiac contractions that result from such signals, any of which may generally be termed “cardiac events.” In at least some examples, MD 100 may additionally include features that allow MD 100 to sense other physical parameters (e.g. mechanical contraction, heart sounds, blood pressure, blood-oxygen levels, etc.) of the heart. MD 100 may include the ability to determine a heart rate and/or occurrences of arrhythmias based on the sensed cardiac events or other physiological parameters.

In some examples, MD 100 may be able to deliver electrical stimulation to the heart in order to ensure synchronized contractions or to treat any detected arrhythmias. Some example arrhythmias include un-synchronized contractions between the atria and ventricles of the heart, bradyarrhythmias, tachyarrhythmias, and fibrillation. For example, MD 100 may be configured to deliver electrical stimulation, such as pacing pulses, defibrillation pulses, or the like, in order to implement one or more therapies. Some example of such therapies may include multi-chamber therapy, e.g. therapy to ensure synchronized contraction of the various chambers of the heart, bradycardia therapy, ATP therapy, CRT, defibrillation, or other electrical stimulation therapies in order to treat one or more arrhythmias. In some examples, MD 100 may coordinate with one or more separate devices in order to deliver one or more therapies.

FIG. 1 is an illustration of one example medical device 100. The illustrative MD 100 may include a sensing module 102, a pulse generator module 104, a processing module 106, a telemetry module 108, and a battery 110, all housed within a housing 120. MD 100 may further include leads 112, and electrodes 114 attached to housing 120 and in electrical communication with one or more of the modules 102, 104, 106, and 108 housed within housing 120.

Leads 112 may be connected to and extend away from housing 120 of MD 100. In some examples, leads 112 are implanted on or within the heart of the patient, such as heart 115. Leads 112 may contain one or more pacing electrodes 114 positioned at various locations on leads 112 and distances from housing 120. Some leads 112 may only include a single pacing electrode 114 while other leads 112 may include multiple pacing 114. Generally, pacing 114 are positioned on leads 112 such that when leads 112 are implanted within the patient, one or more pacing electrodes 114 are in contact with the patient's cardiac tissue. Accordingly, electrodes 114 may conduct received cardiac electrical signals to leads 112. Leads 112 may, in turn, conduct the received cardiac electrical signals to one or more modules 102, 104, 106, and 108 of MD 100. In a similar manner, MD 100 may generate electrical stimulation, and leads 112 may conduct the generated electrical stimulation to pacing electrodes 114. Pacing electrodes 114 may then conduct the generated electrical stimulation to the cardiac tissue of the patient. When discussing sensing cardiac electrical signals and delivering generated electrical stimulation, this disclosure may consider such conduction implicit in those processes.

Sensing module 102 may be configured to sense the cardiac electrical events. For example, sensing module 102 may be connected to leads 112 and pacing electrodes 114 through leads 112 and sensing module 102 may be configured to receive cardiac electrical signals, e.g. cardiac events, conducted through pacing electrodes 114 and leads 112. In some examples, leads 112 may include various sensors, such as accelerometers, blood pressure sensors, heart sound sensors, blood-oxygen sensors, and other sensors which measure physiological parameters of the heart and/or patient. In other examples, such sensors may be connected directly to sensing module 102 rather than to leads 112. In any case, sensing module 102 may be configured to receive such signals produced by any sensors connected to sensing module 102, either directly or through leads 112. Sensing module 102 may additionally be connected to processing module 106 and may be configured to communicate such received signals to processing module 106. In some examples, sensing module 102 is configured to sense cardiac electrical events from only the chamber in which MD 100 is affixed. In other examples, sensing module 102 is configured to sense cardiac electrical events from the chamber in which MD 100 is affixed and from other chambers of heart 110.

Pulse generator module 104 may be connected to pacing electrodes 114. In some examples, pulse generator module 104 may be configured to generate electrical stimulation signals to provide electrical stimulation to the heart. For example, pulse generator module 104 may generate such electrical stimulation signals by using energy stored in battery 110 within MD 100. Pulse generator module 104 may be configured to generate electrical stimulation signals in order to provide one or multiple of a number of different therapies. For example, pulse generator module 104 may be configured to generate electrical stimulation signals, such as pacing pulses or the like, to provide multi-chamber therapies, bradycardia therapy, tachycardia therapy, cardiac resynchronization therapy, and fibrillation therapy. Multi-chamber therapies may include techniques for detecting un-synchronized contractions of the heart and coordinating a delivery of pacing pulses to the various chambers of the heart in order to ensure synchronization of contractions. Bradycardia therapy may include generating and delivering pacing pulses at a rate faster than the intrinsically generated electrical signals in order to try to increase the heart rate. Tachycardia therapy may include ATP therapy. Cardiac resynchronization therapy (CRT) may include delivering electrical stimulation to ventricles of the heart in order to produce a more efficient contraction of the ventricles. Fibrillation therapy may include delivering a fibrillation pulse to try to override the heart and stop the fibrillation state. In other examples, pulse generator 104 may be configured to generate electrical stimulation signals to provide different electrical stimulation therapies to treat one or more detected arrhythmias and/or other heart conditions.

Processing module 106 can be configured to control the operation of MD 100. For example, processing module 106 may be configured to receive electrical signals from sensing module 102. Based on the received signals, processing module 106 may be able to determine a heart rate. In at least some examples, processing module 106 may be configured to determine occurrences of arrhythmias, based on the heart rate, various features of the received signals, or both. Based on any determined arrhythmias, processing module 106 may be configured to control pulse generator module 104 to generate electrical stimulation in accordance with one or more therapies to treat the determined one or more arrhythmias. Processing module 106 may further receive information from telemetry module 108. In some examples, processing module 106 may use such received information in determining whether an arrhythmia is occurring or to take particular action in response to the information. Processing module 106 may additionally control telemetry module 108 to send information to other devices.

In some examples, processing module 106 may include a pre-programmed chip, such as a very-large-scale integration (VLSI) chip or an application specific integrated circuit (ASIC). In such embodiments, the chip may be pre-programmed with control logic in order to control the operation of MD 100. By using a pre-programmed chip, processing module 106 may use less power than other programmable circuits while able to maintain basic functionality, thereby increasing the battery life of MD 100. In other examples, processing module 106 may include a programmable microprocessor. Such a programmable microprocessor may allow a user to adjust the control logic of MD 100, thereby allowing for greater flexibility of MD 100 than when using a pre-programmed chip. In some examples, processing module 106 may further include a memory circuit and processing module 106 may store information on and read information from the memory circuit. In other examples, MD 100 may include a separate memory circuit (not shown) that is in communication with processing module 106, such that processing module 106 may read and write information to and from the separate memory circuit.

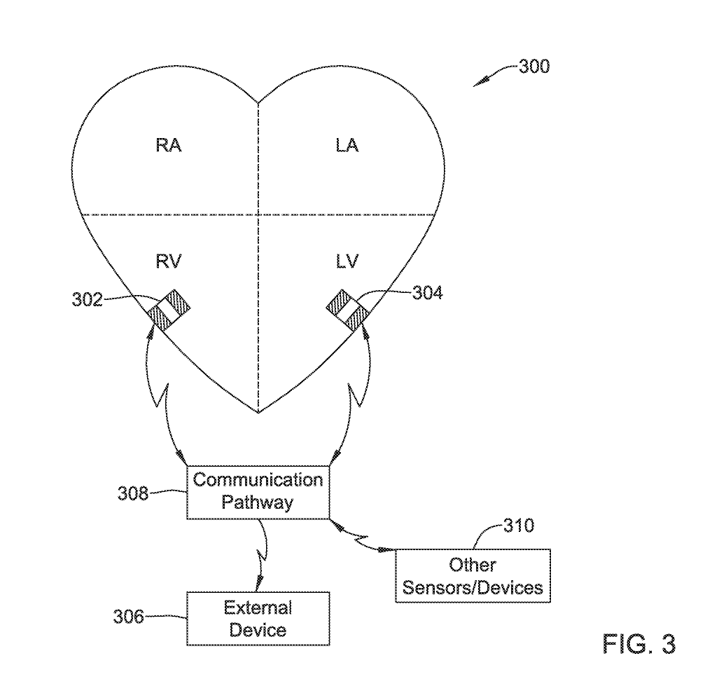

Telemetry module 108 may be configured to communicate with devices such as sensors, other medical devices, or the like, that are located externally to MD 100. Such devices may be located either external or internal to the patient's body. Irrespective of the location, external devices (i.e. external to the MD 100 but not necessarily external to the patient's body) can communicate with MD 100 via telemetry module 108 to accomplish one or more desired functions. For example, MD 100 may communicate sensed electrical signals to an external medical device through telemetry module 108. The external medical device may use the communicated electrical signals in determining a heart rate and/or occurrences of arrhythmias or in coordinating its function with MD 100. MD 100 may additionally receive sensed electrical signals from the external medical device through telemetry module 108, and MD 100 may use the received sensed electrical signals in determining a heart rate and/or occurrences of arrhythmias or in coordinating its function with MD 100. Telemetry module 108 may be configured to use one or more methods for communicating with external devices. For example, telemetry module 108 may communicate via radiofrequency (RF) signals, inductive coupling, optical signals, acoustic signals, conducted communication signals, or any other signals suitable for communication. Communication techniques between MD 100 and external devices will be discussed in further detail with reference to FIG. 3 below.

Battery 110 may provide a power source to MD 100 for its operations. In one example, battery 110 may be a non-rechargeable lithium-based battery. In other examples, the non-rechargeable battery may be made from other suitable materials known in the art. Because, in examples where MD 100 is an implantable device, access to MD 100 may be limited, it is necessary to have sufficient capacity of the battery to deliver sufficient therapy over a period of treatment such as days, weeks, months, or years. In other examples, battery 110 may a rechargeable lithium-based battery in order to facilitate increasing the useable lifespan of MD 100.

In some examples, MD 100 may be an implantable cardiac pacemaker (ICP). In such an example, MD 100 may have one or more leads, for example leads 112, which are implanted on or within the patient's heart. The one or more leads 112 may include one or more pacing electrodes 114 that are in contact with cardiac tissue and/or blood of the patient's heart. MD 100 may also be configured to sense cardiac events and determine, for example, a heart rate and/or one or more cardiac arrhythmias, based on analysis of the sensed cardiac events. MD 100 may further be configured to deliver multi-chamber therapy, CRT, ATP therapy, bradycardia therapy, defibrillation therapy and/or other therapy types via leads 112 implanted within the heart. In at least some examples, MD 100 may be configured to deliver therapy separately to multiple chambers of the heart, either alone or in combination with one or more other devices.

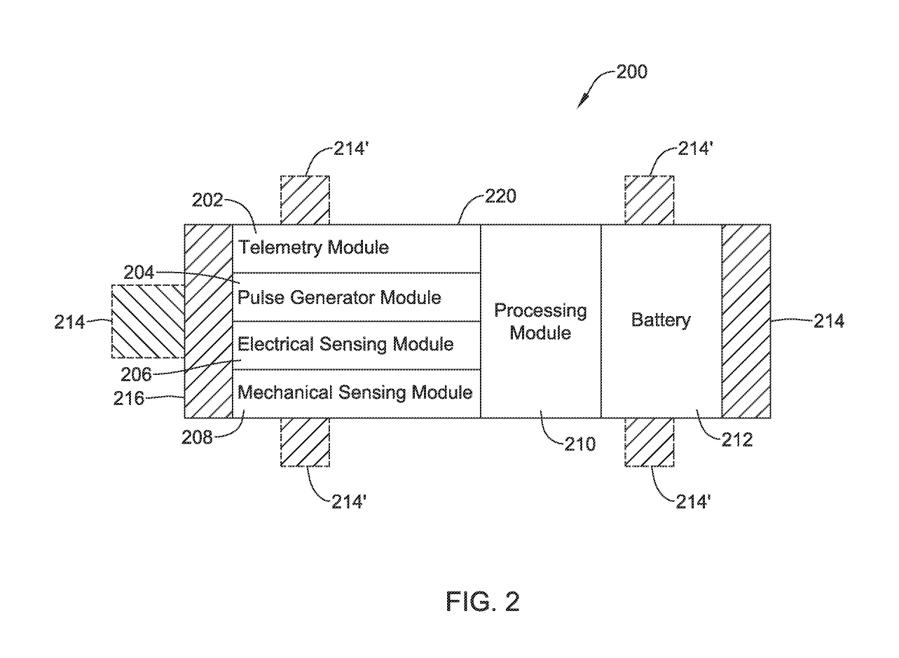

In other examples, MD 100 may be a leadless cardiac pacemaker (LCP—described more specifically with respect to FIG. 2). In such examples, MD 100 may not include leads 112 that extend away from housing 120. Rather, MD 100 may include pacing electrodes 114 coupled relative to the housing 120. In these examples, MD 100 may be implanted on or within the patient's heart at a desired location.

FIG. 2 is an illustration of an exemplary leadless cardiac pacemaker (LCP) 200. In the example shown, LCP 200 may include all of the modules and components of MD 100, except that LCP 200 may not include leads 112. As can be seen in FIG. 2, LCP 200 may be a compact device with all components housed within LCP 200 or directly on housing 220. As illustrated in FIG. 2, LCP 200 may include telemetry module 202, pulse generator module 204, processing module 210, and battery 212. Such components may have a similar function to the similarly named modules and components as discussed in conjunction with MD100 of FIG. 1.

In some examples, LCP 200 may include electrical sensing module 206 and mechanical sensing module 208. Electrical sensing module 206 may be similar to sensing module 102 of MD 100. For example, electrical sensing module 206 may be configured to sense or receive cardiac events. Electrical sensing module 206 may be in electrical connection with pacing electrodes 214 and/or 214′, which may conduct the cardiac events to electrical sensing module 206. Mechanical sensing module 208 may be configured to receive one or more signals representative of one or more physiological parameters of the heart. For example, mechanical sensing module 208 may include, or be in electrical communication with one or more sensors, such as accelerometers, blood pressure sensors, heart sound sensors, blood-oxygen sensors, and other sensors which measure physiological parameters of the patient. Although described with respect to FIG. 2 as separate sensing modules, in some examples, electrical sensing module 206 and mechanical sensing module 208 may be combined into a single module.

In at least one example, each of modules 202, 204, 206, 208, and 210 illustrated in FIG. 2 may be implemented on a single integrated circuit chip. In other examples, the illustrated components may be implemented in multiple integrated circuit chips that are in electrical communication with one another. All of modules 202, 204, 206, 208, and 210 and battery 212 may be encompassed within housing 220. Housing 220 may generally include any material that is known as safe for implantation within a human body and may hermetically seal modules 202, 204, 206, 208, and 210 and battery 212 from fluids and tissues when LCP 200 is implanted within a patient.

As depicted in FIG. 2, LCP 200 may include pacing electrodes 214, which can be secured relative to housing 220 but exposed to the tissue and/or blood surrounding the LCP 200. As such, pacing electrodes 214 may be generally disposed on either end of LCP 200 and may be in electrical communication with one or more of modules 202, 204, 206, 208, and 210. In some examples, pacing electrodes 214 may be connected to housing 220 only through short connecting wires such that electrodes pacing 214 are not directly secured relative to housing 220. In some examples, LCP 200 may additionally include one or more electrodes pacing 214′. Pacing electrodes 214′ may be positioned on the sides of LCP 200 and increase the number of pacing electrodes by which LCP 200 may sense cardiac electrical activity and/or deliver electrical stimulation. Pacing electrodes 214 and/or 214′ can be made up of one or more biocompatible conductive materials such as various metals or alloys that are known to be safe for implantation within a human body. In some instances, pacing electrodes 214 and/or 214′ connected to LCP 200 may have an insulative portion that electrically isolates the pacing electrodes 214 from, adjacent electrodes, the housing 220, and/or other materials.

To implant LCP 200 inside patient's body, an operator (e.g., a physician, clinician, etc.), may need to fix LCP 200 to the cardiac tissue of the patient's heart. To facilitate fixation, LCP 200 may include one or more anchors 216. Anchor 216 may be any one of a number of fixation or anchoring mechanisms. For example, anchor 216 may include one or more pins, staples, threads, screws, helix, tines, and/or the like. In some examples, although not shown, anchor 216 may include threads on its external surface that may run along at least a partial length of anchor 216. The threads may provide friction between the cardiac tissue and the anchor to help fix anchor 216 within the cardiac tissue. In other examples, anchor 216 may include other structures such as barbs, spikes, or the like to facilitate engagement with the surrounding cardiac tissue.

The design and dimensions of MD 100 and LCP 200, as shown in FIGS. 1 and 2, respectively, can be selected based on various factors. For example, if the medical device is for implant on the endocardial tissue, such as is sometimes the case of an LCP, the medical device can be introduced through a femoral vein into the heart. In such instances, the dimensions of the medical device may be such as to be navigated smoothly through the tortuous path of the vein without causing any damage to surrounding tissue of the vein. According to one example, the average diameter of the femoral vein may be between about 4 mm to about 8 mm in width. For navigation to the heart through the femoral vein, the medical device can have a diameter of at less than 8 mm. In some examples, the medical device can have a cylindrical shape having a circular cross-section. However, it should be noted that the medical device can be made of any other suitable shape such as rectangular, oval, etc. A flat, rectangular-shaped medical device with a low profile may be desired when the medical device is designed to be implanted subcutaneously.

FIGS. 1 and 2 above described various examples of implantable medical devices. In some examples, a medical device system may include more than one medical device. For example, multiple medical devices 100/200 may be used cooperatively to detect and treat cardiac arrhythmias and/or other cardiac abnormalities. For example, multiple medical devices may be implanted in multiple chambers of the heart to provide multi-chamber therapy. Some example systems will be described below in connection with FIGS. 3-6. In such multiple device systems, it may be desirable to have the medical devices communicate with each other, or at least have some of the devices receive communication signals from other medical devices. Some example communication techniques are described below with respect to FIG. 3.

FIG. 3 illustrates an example of a medical device system and a communication pathway via which multiple medical devices may communicate. In the example shown, medical device system 300 may include LCPs 302 and 304, external medical device 306, and other sensors/devices 310. External device 306 may be any of the devices described previously with respect to MD 100, in addition to other medical devices such as implantable cardioverter-defibrillators (ICDs), diagnostic only medical devices, or other implanted or external (e.g. external to a patient's body) medical devices. Other sensors/devices 310 may also be any of the devices described previously with respect to MD 100 or other medical devices such as ICDs, diagnostic only devices, or other suitable medical devices. In other examples, other sensors/devices 310 may include a sensor, such as an accelerometer or blood pressure sensor, or the like. In still other examples, other sensors/devices 310 may include an external programmer device that may be used to program one or more devices of system 300.

Various devices of system 300 may communicate via communication pathway 308. For example, LCPs 302 and/or 304 may sense cardiac events, for example intrinsically generated or conducted signals, and may communicate such signals or information relating to such signals to one or more other devices 302/304, 306, and 310 of system 300 via communication pathway 308. In one example, external device 306 may receive the communicated signals and, based on the received signals, determine a heart rate and/or an occurrence of an arrhythmia. In some cases, external device 306 may communicate such determinations to one or more other devices 302/304, 306, and 310 of system 300. In other examples, LCPs 302 and 304 may determine heart rates or arrhythmias based on the communicated signals and may communicate such determinations to other communicatively coupled devices. Additionally, one or more other devices 302/304, 306, and 310 of system 300 may take action based on the communications, such as by delivering suitable electrical stimulation.

Communication pathway 308 may represent one or more of various communication methods. For example, the devices of system 300 may communicate with each other via RF signals, inductive coupling, optical signals, acoustic signals, or any other signals suitable for communication and communication pathway 308 may represent such signals.

In at least one example, communicated pathway 308 may represent conducted communication signals. Accordingly, devices of system 300 may have components that allow for conducted communication. In examples where communication pathway 308 includes conducted communication signals, devices of system 300 may communicate with each other by delivering electrical communication pulses into the patient's body by one device of system 300. The patient's body may conduct these electrical communication pulses and other devices of system 300 may sense such conducted communication pulses. In such examples, the delivered electrical communication pulses may differ from the electrical stimulation pulses of any of the above described electrical stimulation therapies. For example, the devices of system 300 may deliver such electrical communication pulses at a voltage level that is sub-threshold. That is, the voltage amplitude of the delivered electrical communication pulses may be low enough as to not capture the heart (e.g. not cause a contraction). Although, in some circumstances, one or more delivered electrical communication pulses may, deliberately or inadvertently capture the heart, and in other circumstances, delivered electrical stimulation may not capture the heart. In some cases, the delivered electrical communication pulses may be modulated (e.g. pulse width or amplitude modulated), or the timing of the delivery of the communication pulses may be modulated, to encode the communicated information. These are just some examples of how varying parameters of the communication pulse may convey information to another device. Other techniques may be used with such a conducted communication technique.

As mentioned above, some example systems may employ multiple devices for determining occurrences of arrhythmias and/or other heart conditions, and/or for delivering electrical stimulation. FIGS. 3-6 describe various example systems that may use multiple devices in order to determine occurrences of arrhythmias and/or deliver electrical stimulation therapy. However, FIGS. 3-6 should not be viewed as limiting examples. For example, FIGS. 3-6 describe how various multiple device systems may coordinate to detect various arrhythmias and/or other heart conditions, and/or deliver electrical stimulation therapy. In general, any combinations of devices such as that described with respect to MD 100 and LCP 200 may used in concert with the below described techniques for detecting arrhythmias and/or other heart conditions, and/or delivering electrical stimulation therapy.

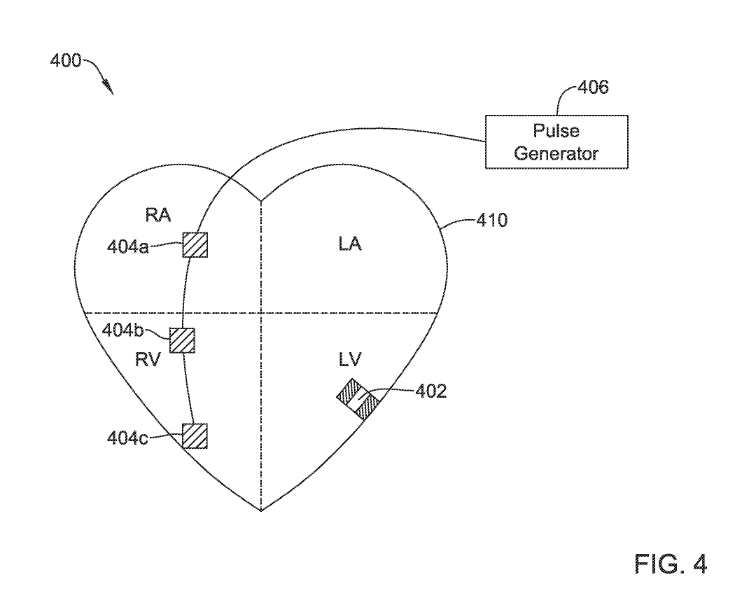

FIG. 4 illustrates an example medical device system 400 that includes an LCP 402 and a pulse generator 406. In this example, pulse generator 406 may be an implantable cardiac pacemaker (ICP). For example, pulse generator 406 may be an ICP such as that described previously with respect to MD 100. In examples where pulse generator 406 is an ICP, pacing electrodes 404a, 404b, and 404c may be implanted on or within the right ventricle and/or right atrium of heart 410 via one or more leads. In other contemplated examples, pulse generator 406 may include pacing electrodes implanted in the left ventricle and/or atrium of heart 410. These pacing electrodes may instead be of or in addition to electrodes implanted within the right ventricle and/or atrium of heart 410.

As shown, an LCP 402 may be implanted within heart 410. Although LCP 402 is depicted implanted within the left ventricle (LV) of the heart 410, in some instances, LCP 402 may be implanted within a different chamber of the heart 410. For example, LCP 402 may be implanted within the left atrium (LA) of heart 410 or the right atrium (RA) of heart 410. In other examples, LCP 502 may be implanted within the right ventricle (RV) of heart 410.

In any event, LCP 402 and pulse generator 406 may operate together to detect cardiac events and deliver electrical stimulation therapy. In some examples, devices 402 and 406 may operate independently to sense cardiac events of heart 410. For example, LCP 402 may sense cardiac events in the LV of heart 410 while pulse generator 406 may sense cardiac events in the RA and/or RV of heart 410. Either or both devices may optionally determine a contraction rate or occurrence of an arrhythmia based on the sensed cardiac events. In some examples, the contraction rate may be a rate of sensed cardiac events. That is, LCP 402 may determine a contraction rate for the LV of heart 410 while pulse generator 406 may determine a contraction rate for the RA and/or RV of heart 410. In some examples, devices 402 and 406 may determine occurrences of arrhythmias based at least in part on these determined contraction rates.

In some examples, devices 402 and 406 may additionally send and/or receive communication signals in order to more effectively deliver electrical stimulation to heart 410. For example, LCP 402 may send indications of cardiac events sensed in the LV to pulse generator 406 and pulse generator 406 may send indications of cardiac events sensed in the RA and/or RV to LCP 402. Devices 402 and 406 may additionally communicate any determined contraction rates to the other device. In some examples, devices 402 and 406 may optionally or additionally communicate other signals such as commands to perform various actions, for example to deliver electrical stimulation to heart 410. As described above, devices 402 and 406 may utilize one or a number of communication pulses to convey such information. In some examples, communication may only occur in one direction. That is only one of devices 402 and 406 may send communication signals to the other of devices 402 and 406. The receiving device may then make one or more determinations, such as contraction rate determinations or arrhythmia determinations, based on the received signals. Alternatively, the receiving device may perform one or more actions based on the received communication signals, for example delivering electrical stimulation.

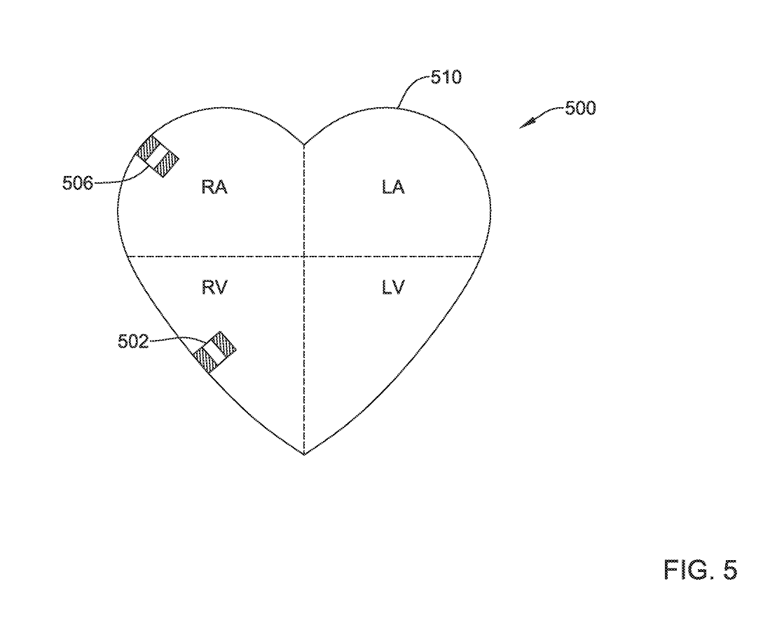

FIG. 5 illustrates an example medical device system 500 that includes LCP 502 and LCP 506. LCP 502 and LCP 506 are shown implanted within a heart 510. Although LCPs 502 and 506 are depicted as implanted within the right ventricle (RV) of heart 510 and right atrium (RA) of heart 510, respectively, in other examples, LCPs 502 and 506 may be implanted within different chambers of heart 510. For example, system 500 may include LCPs 502 and 506 implanted within both atria of heart 510. In other examples, system 500 may include LCPs 502 and 506 implanted within both ventricles of heart 510. In more examples, system 500 may include LCPs 502 and 506 implanted within any combination of ventricles and atria. In yet other examples, system 500 may include LCPs 502 and 506 implanted within the same chamber of heart 510.

In any event, LCP 502 and LCP 506 may operate together to detect cardiac events and deliver electrical stimulation therapy. In some examples, devices 502 and 506 may operate independently to sense cardiac events of heart 510. For example, LCP 502 may sense cardiac events in the RV of heart 510 while LCP 506 may sense cardiac events in the RA of heart 510. Either or both devices may optionally determine a contraction rate or occurrence of an arrhythmia based on the sensed cardiac events. In some examples, the contraction rate may be a rate of sensed cardiac events. That is, LCP 502 may determine a contraction rate for the RV of heart 510 while LCP 506 may determine a contraction rate for the RA of heart 510. In some examples, devices 502 and 506 may determine occurrences of arrhythmias based at least in part on these determined contraction rates.

In some examples, devices 502 and 506 may additionally send and/or receive communication signals in order to more effectively deliver electrical stimulation to heart 510. For example, LCP 502 may send indications of cardiac events sensed in the RV to LCP 506 and LCP 506 may send indications of cardiac events sensed in the RA to LCP 502. Devices 502 and 506 may additionally communicate any determined contraction rates to the other device. In some examples, devices 502 and 506 may optionally or additionally send other signals such as commands to perform various actions, for example to deliver electrical stimulation to heart 510. In some examples, communication may only occur in one direction. That is only one of devices 502 and 506 may send communication signals to the other of devices 502 and 506. The receiving device may then make one or more determinations, such as contraction rate determinations or arrhythmia determinations, based on the received signals. Alternatively, the receiving device may perform one or more actions based on the received communication signals, for example delivering electrical stimulation.

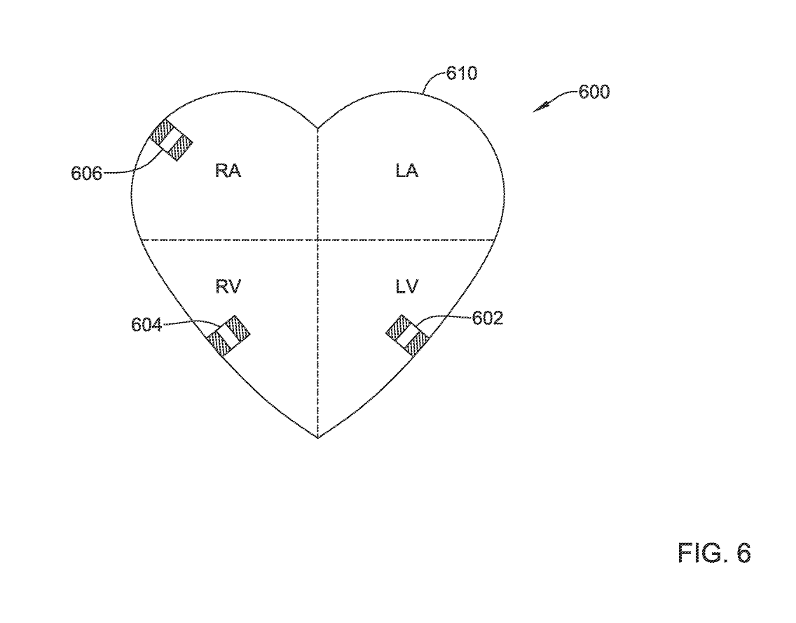

FIG. 6 illustrates an example medical device system 600 with three separate LCPs including LCP 602, LCP 604, and LCP 606. Although system 600 is depicted with LCPs 602, 604, and 606 implanted within the LV, RV, and RA, respectively, other examples may include LCPs 602, 604, and 606 implanted within different chambers of the heart 610. For example, system 600 may include LCPs implanted within both atria and one ventricle of the heart 610. In other examples, system 600 may include LCP 606 implanted within the LA of heart 610. More generally, it is contemplated that system 600 may include LCPs implanted within any combination of ventricles and atria. In some instances, system 600 may include two or more of LCPs 602, 604, and 606 implanted within the same chamber of the heart 610.

In any event, LCPs 602, 604, and 606 may operate together to detect cardiac events and deliver electrical stimulation therapy. In some examples, devices 602, 604, and 606 may operate independently to sense cardiac events of heart 610. For example, LCP 602 may sense cardiac events in the LV of heart 610, LCP 604 may sense cardiac events in the RV of heart 610, and LCP 606 may sense cardiac events in the RA of heart 610. Any or all of devices 602, 604, and 606 may optionally determine a contraction rate or occurrence of an arrhythmia based on the sensed cardiac events. In some examples, the contraction rate may be a rate of sensed cardiac events. That is, LCP 602 may determine a contraction rate for the LV of heart 610, LCP 604 may determine a contraction rate for the RB of heart 610, and LCP 606 may determine a contraction rate for the RA of heart 610. In some examples, devices 602, 604, and 606 may determine occurrences of arrhythmias based at least in part on these determined contraction rates.

In some examples, devices 602, 604, and 606 may additionally send and/or receive communication signals in order to more effectively deliver electrical stimulation to heart 610. For example, LCP 602 may send indications of cardiac events sensed in the LV to LCPs 604 and 606, LCP 604 may send cardiac events sensed in the RV to LCPs 602 and 606, and LCP 606 may send indications of cardiac events sensed in the RA to LCPs 602 and 604. Devices 602, 604, and 606 may additionally communicate any determined contraction rates to the other devices. In some examples, devices 602, 604, and 606 may optionally or additionally send other signals such as commands to perform various actions, for example to deliver electrical stimulation to heart 610. In some examples, some of devices 602, 604, and 606 may only be configured to receive communication signals while others of devices 602, 604, and 606 may only be configured to send communication signals. For instance, only one or two of devices 602, 604, and 606 may only be configured to send communication signals. Additionally in some examples, only one or two of devices 602, 604, and 606 may only be configured to receive communication signals. In at least some examples, at least one of devices 602, 604, and 606 may be configured to both send and receive communication signals. Any of the receiving devices may then make one or more determinations, such as contraction rate determinations or arrhythmia determinations, based on the received signals. Alternatively, the receiving devices may perform one or more actions based on the received communication signals, for example delivering electrical stimulation.

The above described multi-device systems should not be construed as limiting the disclosed techniques to any particular multi-device configuration. As one example, one system may include two LCP devices and one ICP device. In other examples, some multi-device systems may include more than three devices, for instance systems may comprise four LCP devices or three LCP devices and an ICP device. Even the spatial positions of the LCPS and/or electrodes of the ICP as depicted in FIG. 3-6 are merely exemplary. For example, the LCPs may not reside within the chambers of the heart. Rather, in some examples, one or more of the LCPs may reside on an epicardial surface of the heart proximate a chamber of the heart. The electrodes of the ICP may vary in number and/or may span more or fewer chambers in some examples. Accordingly, many variations of the depicted multi-device systems are contemplated that may implement the disclosed sensing, treatment, and communication techniques described herein.

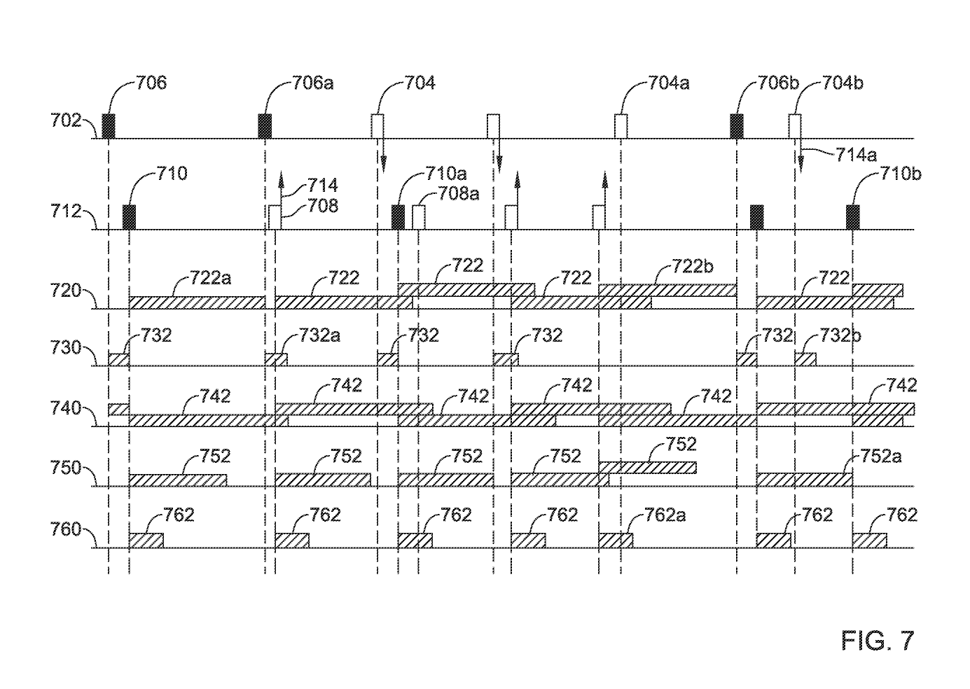

FIG. 7 depicts a communication technique for use with a medical device system comprising at least two implantable medical devices (IMDs), such as MD 100/LCP 200 or two LCPs. Time lines 702 and 712 of FIG. 7 show illustrative sensed cardiac events, paced cardiac events, and communication signals. For example, time line 702 shows illustrative sensed atrial cardiac events 704 sensed by a first IMD implanted within or proximate an atrium of a heart. Time line 702 also includes paced atrial cardiac events 706, which represent a delivery of electrical stimulation, e.g. a pacing pulse, by the first IMD and a corresponding contraction of the atrium in response to the delivered electrical stimulation. Time line 712 depicts sensed ventricular cardiac events 708 and paced ventricular cardiac events 710 corresponding to a second IMD implanted within or proximate a ventricle of the heart. In FIG. 7, open bars represent sensed cardiac events, for example, sensed atrial events 704, and closed bars represent paced cardiac events, such as paced ventricular events 706. FIG. 7 also depicts communication signals 714, shown as arrows. A communication signal 714 on time line 702 represents a communication from the first IMD to the second IMD, and a communication signal 714 depicted on time line 712 represents a communication from the second IMD to the first IMD.

In the example shown in FIG. 7, communication signals 714 occur mostly in combination with sensed atrial cardiac events 704 and sensed ventricular cardiac events 708. In at least some examples, the first and second IMDs may be configured to sense the paced events corresponding to the other IMD, and thus the pacing pulse itself functions as both a pacing pulse and a communication signal. For example, the second IMD may be able to sense paced atrial cardiac events 706, and the first IMD and may be able to sense paced ventricular cardiac events 710. Accordingly, by not sending communication signals 714 in conjunction with the paced cardiac events, the system may save energy. However, in some examples, the first and/or second IMDs may additionally send communication signals in conjunction paced cardiac events, for example as a safety measure. As used herein, the term “communicated events” may encompass both cardiac events communicated by communication signals 714 that are separate from pacing pulses, and paced cardiac events which may not be indicated by separate communication signals 714, as both may communicate information about a cardiac event from one IMD to the other IMD. Additionally, “atrial communicated events” may encompass both sensed atrial cardiac events 704 which may be communicated to the second IMD (as indicated by communication signals 714 which may be separate from pacing pulses) and paced atrial cardiac events 706. Likewise, “ventricular communicated events” may encompass both sensed ventricular cardiac events 708 which may be communicated to the first IMD (as indicated by communication signals 714 that may be separate from pacing pulses) and paced ventricular cardiac events 710.

Time lines 720, 730, 740, 750, and 760 all depict predefined periods of time that the first and/or second IMDs may identify often from one or more triggers. The various periods of time may operate to, at least partially, control when the first and/or second IMDs communicate sensed cardiac events and deliver electrical stimulation therapy. Each of the periods of time, and their effect on the system, will be described in more detail below.

FIG. 7 depicts one example of a communication technique whereby the first and second IMDs may coordinate delivery of electrical stimulation therapy. For example, the first IMD may be configured to only selectively communicate sensed atrial cardiac events 704 to the second IMD. Additionally, the second IMD may be configured to only selectively communicate sensed ventricular cardiac events 708 to the first IMD. In at least some examples, the first IMD may only communicate those sensed atrial cardiac events 704 that occur outside of a predetermined time period following each ventricular event, e.g. each sensed ventricular cardiac event 708 and each paced ventricular cardiac event 710. Such a predefined period may be termed a post ventricular atrial refractory period (PVARP), and each PVARP 762 is tracked along time line 760. As one example in FIG. 7, the fifth atrial event 704a on time line 702 falls within a PVARP 762a and, accordingly, the first IMD does not communicate the sensed atrial cardiac event 704a to the second IMD, as evidenced by a lack of a communication signal 714 associated with the fifth atrial event 704a. As seen in FIG. 7, each sensed ventricular cardiac event 708 and paced ventricular cardiac event 710 begins a new PVARP 762. The use of a PVARP 762 may better help to coordinate contractions between the atria and ventricles of the heart.

Additionally or optionally in other examples, the second IMD may only communicate sensed ventricular cardiac events 708 that occur outside of a predefined time period after the last sensed ventricular cardiac event 708 or paced ventricular cardiac event 710. For example, the fourth ventricular cardiac event of FIG. 7, a sensed ventricular cardiac event 708a, occurs very close in time to the third cardiac event 710a. Accordingly, the second IMD may not send a communication signal 714 corresponding to the sensed ventricular cardiac event 708a to the first IMD. Sensed ventricular cardiac events 708 that occur close in time to other sensed ventricular cardiac events 708 or paced ventricular cardiac events 710 are likely to be noise or other artifacts which do not represent actual cardiac events. Accordingly, limiting the communication of such events helps to ensure the system is responding to actual cardiac functions. Although this feature is described with respect to the second IMD, in some examples the first IMD may include a similar feature that limits communicating atrial cardiac events that occur closely in time to other atrial cardiac events.

FIG. 7 also depicts coordination of communicated cardiac events and the delivery of electrical stimulation therapy by the first and second IMDs. For example, the second IMD may be configured to deliver a pacing pulse to the ventricle of the heart in response to a communicated event. In some examples, the second IMD may be configured to deliver a pacing pulse at the expiration of a predefined time period, sometimes termed an atrio-ventricular delay (AV) delay period. The second IMD may track an AV delay period 732 after each atrial communicated event, and each AV delay period 732 may be tracked along a time line 730. The second IMD may additionally be configured to only deliver a pacing pulse if the second IMD does not sense an intrinsic ventricular cardiac event (e.g. represented by sensed ventricular cardiac events 708) that occurs within the AV delay period 732. For example, the second ventricular cardiac event 708 on time line 712 occurs within the AV delay period 732a. Accordingly, the second IMD does not also deliver a pacing pulse to the heart, which would otherwise result in a paced ventricular cardiac event 710.

In a similar manner, the first IMD may track a ventricular-atrial (VA) delay period 722. The first IMD may track a VA delay period 722 after each ventricular communicated event, and each VA delay period 722 may be tracked along a time line 720. At the expiration of each VA delay period 722, the first IMD may be configured to deliver a pacing pulse to the atrium of the heart. However, if the first IMD senses an intrinsic atrial event (e.g. represented by sensed atrial cardiac events 704) during such VA delay period 722, the first IMD may be configured to not deliver a pacing pulse at the expiration of the VA delay period 722 and may instead wait to start a new VA delay 722 period after the next ventricular communicated event. Some examples may include one or more exceptions. For instance, the first IMD may ignore any sensed atrial cardiac events 704 that occur within a PVARP 762 for the purposes of determining whether to deliver a pacing pulse at the expiration of a VA delay period 722. For example, the second atrial cardiac event 706a of time line 702 is a paced atrial cardiac event which occurs at the expiration of a VA delay period 722a. This second atrial event 706a represents a pacing pulse delivered by the first IMD in response to the expiration of the VA delay period 722a. As another example, the fifth atrial cardiac event 704a of time line 702 occurs within a PVARP 762a. Accordingly, the first IMD may ignore this atrial cardiac event for purposes of determining whether to deliver a pacing pulse to the atrium of the heart, and the sixth atrial cardiac event 706b, a paced atrial cardiac event, represents the first IMD delivering a pacing pulse to the atrium of the heart at the expiration of the VA delay period 722b.

FIG. 7 also illustrates one or more safety features that may be employed by the system. For example, the second IMD may track two additional predefined time periods, a lower rate limit interval (LRLI) period 742 and a maximum tracking rate interval (MTRI) 752. The LRLI period 742 resets at each sensed ventricular cardiac event 708 and paced ventricular cardiac event 710, and each LRLI period 742 is tracked on time line 740. The second IMD may be configured to deliver a pacing pulse at the expiration of the LRLI period 742. In operation, this LRLI period 742 may result in a minimum contraction rate of the ventricle of the heart, as it helps ensures that the second IMD delivers a pacing pulse at least once every expiration of the LRLI time period. Thus, this LRLI time period 742 may help ensure that the contraction rate of the ventricle never falls to a dangerously low rate. The MTRI period 752 also resets at each sensed ventricular cardiac event 708 and paced ventricular cardiac event 710, and each MTRI period 752 is tracked on time line 750. Unlike the LRLI period 742, the MTRI period 752 sets a maximum rate at which the second IMD may deliver pacing pulses to the ventricle of the heart. For example, the second IMD may be configured to not deliver a pacing pulse until the expiration of the MTRI period 752. This effectively creates a maximum contraction rate of the ventricle of the heart and helps ensure that the contraction rate never increases to a dangerously high level. As one example, the last atrial cardiac event 704b on time line 702 falls within an MTRI period 752a. Although the first IMD communicates the sensed atrial cardiac event 704b, as indicated by the corresponding communication signal 714a, the second IMD does not respond by delivering a pacing pulse at the expiration of an AV delay period 732b. Rather, the second IMD only delivers a pacing pulse at the expiration of the MTRI period 752a, as evidenced by the last ventricular cardiac event 710b on time line 712, a paced ventricular cardiac event.

The above examples described various illustrative features with respect to either the first IMD or the second IMD. However, each of the various features may be implemented by either IMD, and the IMDs may communicate additional signals to help implement these and other features. For example, the first IMD may track the AV delay period 732 rather than the second IMD. In such examples, the first IMD, at the expiration of the AV delay period 732, may send a communication to the second IMD to deliver a pacing pulse. As another example, the second IMD may track the VA delay period 722. As yet another example, the second IMD may track the PVARP 762. In such an example, the first device may still communicate sensed atrial cardiac events to the second IMD, but the second IMD may ignore the communicated atrial cardiac events for the purposes of determining whether the deliver a pacing pulse at the expiration of an MTRI period 752. Accordingly, at the expiration of the VA delay period 722, the second IMD may send a communication to the first IMD to deliver a pacing pulse. In a similar manner, any of the IMDs may track any of the periods and send communications to the other IMD to take action or not to take action according to the timing of the various cardiac events with respect to the time periods.

In some examples, the medical device system may incorporate one or more communication safety features. For example, various of the above described features rely on at least one of the IMDs receiving communicated cardiac events from the other IMD, and in some cases taking action based on those received signals. In instances where the communication system between the IMDs fails, for any of a number of reasons, each of the IMDs may be configured to enter a fall back mode. For example, each IMD may track another period of time that resets whenever the IMD receives a communicated cardiac event, e.g. an indication of a sensed cardiac event. After the expiration of the period of time, the IMD may determine that the communication system has failed and may enter a fall back mode where the IMD operates to independently deliver electrical stimulation based on parameters that are not based on communicated events from the other IMD.

As one example, the second IMD may enter a VVI mode. In the VVI mode, the second IMD may sense ventricular cardiac events, deliver cardiac events to the ventricle, and may be inhibited by sensing ventricular cardiac events. In other words, the second IMD may track a predefined period of time, in some instances similar to the LRLI period described above, which resets after each sensed ventricular event and each paced ventricular event. The second IMD may be configured to deliver a pacing pulse at the expiration of such a predefined time period. In operation, this mode helps ensure that the ventricle of the heart beats at least once per predefined timer period, thereby helping to ensure a minimum heart rate that keeps the heart rate from falling dangerously low. As another example, the first IMD may enter an OOO mode. In the OOO mode, the first IMD may be essentially switched off or in a standby-mode. In the OOO mode, the first IMD may not sense cardiac electrical signals or delivering pacing pulses. Alternatively, the first IMD may fall back into an AAI mode. In an AAI mode, the first IMD may sense atrial cardiac events and deliver pacing pulses to the atrium of the heart. Similarly to the second IMD in the VVI mode, in the AAI mode, the first IMD may track a predetermined period of time that resets after each sensed atrial cardiac event and each paced atrial cardiac event. The first IMD may be configured to deliver a pacing pulse at the expiration of the predefined period of time, thus helping to ensure a minimum atrial contraction rate of the heart.

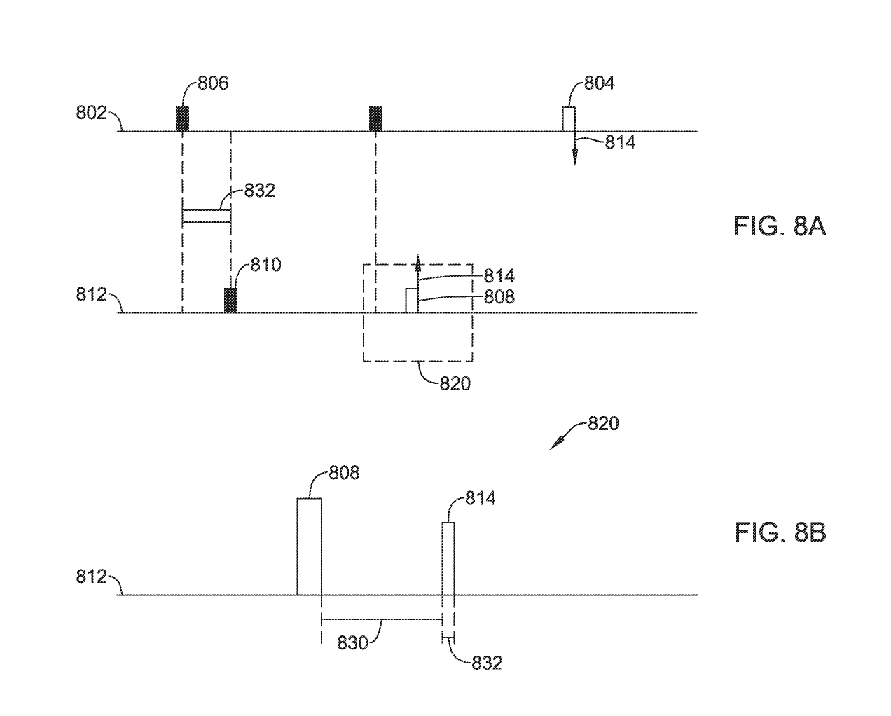

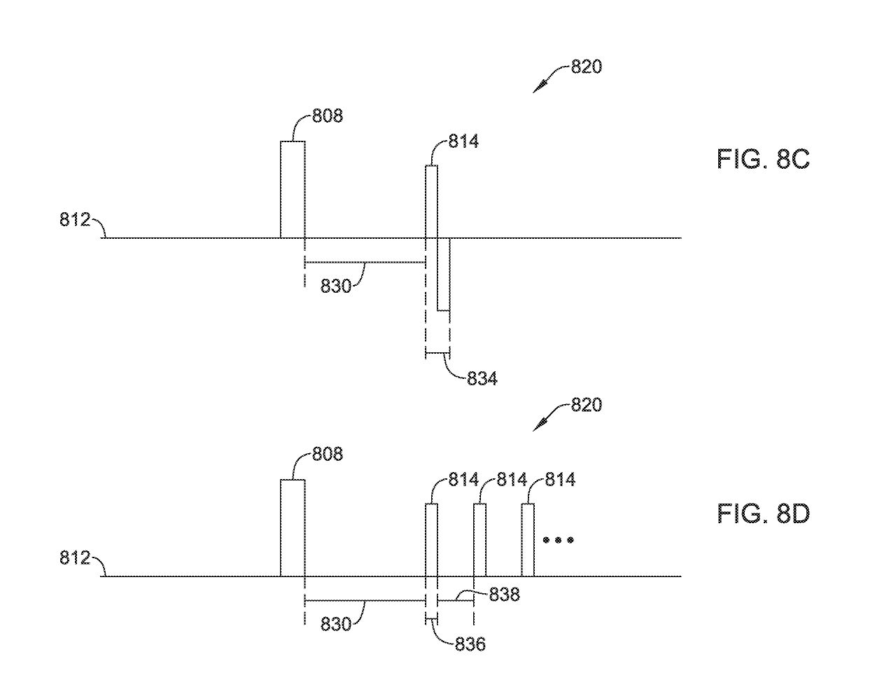

FIGS. 8a-8d depict specific examples of communication pulses that may be used in conjunction with the above described techniques for communication between the first and second IMDs. FIG. 8a depicts a sample graph of atrial and ventricular cardiac events, similar to the graph depicted in FIG. 7. For example, FIG. 8a depicts, on time line 802, sensed atrial cardiac events 804 and paced atrial cardiac events 806, as well as an AV delay period 832. Time line 812 includes sensed ventricular cardiac events 808 and paced ventricular cardiac events 810. Both time lines 802 and 812 include communication signals 814. FIGS. 8b-8d depict region 820 of FIG. 8a in a blown up manner including specifics of communication signal 814 which falls within region 820.

FIG. 8b depicts region 820 including communication signal 814 as a single unipolar pulse. In such examples, the single unipolar pulse may communicate an indication that an IMD sensed a cardiac event to another IMD. In the example of FIGS. 8a-8d, communication signal 814 within region 820 falls on time line 812, which indicates that the second IMD sensed a ventricular cardiac event and sent communication signal 814 to the first IMD. In some examples, the unipolar pulse may have a pulse width 832. Pulse width 832 may be 1 microsecond, 5 microseconds, 10 microseconds, 15 microseconds, or any other suitable pulse width. Additionally, the second IMD may send communication signal 814 a time period 830 after sensed ventricular cardiac event 808. In some examples, time period 830 may be 1 microseconds, 2 microseconds, 5 milliseconds, or any other suitable length of time. In some examples, the first and second IMDs may send communication signals 814 with opposite polarity. In the example of FIG. 8b, the second IMD communicates a positive polarity communication signal 814 and the first IMD communicates a negative polarity communication signal 814, but this is only illustrative. In examples where the first and second IMDs use opposite polarity communication signals, the first IMD may communicate a negative polarity communication signal to indicate a sensed atrial cardiac event. In other examples, the communication signals generated and sent by the different IMDs may vary in different ways, for example by using different pulse widths 832, time period 830, etc.

FIG. 8c depicts region 820 including communication signal 814 as a single bipolar pulse. In such examples, the single bipolar pulse may communicate an indication that an IMD sensed a cardiac event to another IMD. In some examples, the single bipolar pulse may have a pulse width 834. Pulse width 834 may be 2 microseconds, 5 microseconds, 10 microseconds, 15 microseconds, or any other suitable duration. Additionally, in some examples there may be no delay between the phases of the bipolar pulse. In other examples there may be a delay between the phases of the bipolar pulse. The delay may be 1 microseconds, 2 microseconds, 5 microseconds, or any other suitable duration. Additionally, the second IMD may send communication signal 814 a time period 830 after sensed ventricular cardiac event 808. In some examples, time period 830 may be 1 millisecond, 2 milliseconds, 5 milliseconds, or any other suitable length of time. In some examples, the first and second IMDs may send communication signals 814 with opposite polarity. In the example of FIG. 8c, the second device communicated a bipolar pulse with positive polarity followed by negative polarity. In examples where the first and second IMDs use opposite polarity communication signals, the first IMD may communicate a bipolar pulse with negative polarity followed by positive polarity to indicate a sensed atrial cardiac event. In other examples, the communication signals generated and sent by the different IMDs may vary in different ways, for example by using different pulse widths 834, time period 830, etc.

FIG. 8d depicts region 820 including communication signal 814 as a multiple unipolar pulses. In such examples, the multiple unipolar pulses may communicate an indication that an IMD sensed a cardiac event to another IMD. In some examples, each of the multiple unipolar pulses may have a pulse width 836. Pulse width 832 may be 5 microseconds, 10 microseconds, 15 microseconds, or any other suitable length. Additionally, each of the multiple unipolar pulses may be spaced a predetermined period of time 838 away from each other. In some examples, predetermined period of time 838 may be 10 microseconds, 20 microseconds, 30 microseconds, 1 millisecond, 2 milliseconds, or 3 milliseconds, or any other suitable length of time. The second IMD may also send communication signal 814 a time period 830 after sensed ventricular cardiac event 808. In some examples, time period 830 may be 1 millisecond, 2 milliseconds, 5 milliseconds, or any other suitable length of time. In some examples, the first and second IMDs may send communication signals 814 with opposite polarity. In the example of FIG. 8c, the second device communicated positive polarity communication signals 814. In examples where the first and second IMDs use opposite polarity communication signals, the first IMD may communicate negative polarity communication signals to indicate a sensed atrial cardiac event. In other examples, the communication signals generated and sent by the different IMDs may vary in different ways, for example by using different pulse widths 832 or a different predetermined period of time 838. In some examples, each unipolar pulse may represent one bit of information, and multiple unipolar pulses may communicate information in a binary format, for instance by using positive and negative polarity unipolar pulses to represent different bits.

The above descriptions are just some example communications signals that the first and second IMDs may employ for communicating indications of sensed cardiac events and/or other information. In other examples, the first and second IMDs may use different shaped waveforms or spacing schemes for communicating information. By employing any of the above described examples, or a combination of any of the above described examples, the first and second IMDs may help ensure that noise signals received by either of the devices are not improperly interpreted as communication signals. The above described examples may be particularly helpful in embodiments that do not also employ any error checking protocols, for example communication headers, parity bits, cyclic redundancy check (CRC), or other error checking protocols.

The above communication techniques have been described using a system with two IMDs. However, some example communication techniques of the present disclosure may be extended to systems with three or more IMDs. One example communication technique involving three IMDs may be used with system 600 as described above with respect to FIG. 6. For example, a first IMD of the system may be LCP 606 implanted in or proximate the right atrium of heart 610. A second IMD of the system may be LCP 604 implanted in or proximate the right ventricle of heart 610, and a third IMD of the system may be LCP 602 implanted in or proximate the left ventricle of heart 610. LCPs 606 and 604 may be configured according to any of the above disclosed communication techniques. LCP 602 may additionally be configured to receive any communication signals sent by LCPs 606 and/or 604, and sense any delivered pacing pulses delivered by LCPs 606 and 604. In this manner, LCP 602 may be configured to receive any communicated cardiac events from LCPs 606 and 604.

In some examples, communication signals sent by any of LCP 602, 604, and/or 606 may include information that identifies a specific device, if desired. When so provided, devices that receive a communication signal that does not identify the receiving device may ignore that communication signal. In this manner, each device may be able to tailor the communication signals to identify which devices take action based on the communication signal.

LCP 602 may additionally be configured to monitor or track any of the intervals described previously and take action or not take action based on those intervals. For example, LCP 602 may track or monitor an LV LRLI period and deliver a pacing pulse at the expiration of the LV LRLI period. In other examples, LCP 602 may monitor or track a PVARP period, an AV delay period, or any other of the periods described herein.

LCP 602 may additionally be configured to deliver a pacing pulse to heart 610 in or proximate the left ventricle in response to a communicated atrial event. For example, LCP 602 may monitor or track an LV AV delay period. LCP 602 may be configured to track such a period from each communicated atrial event. At the expiration of each LV AV delay period, LCP 602 may be configured to deliver a pacing pulse to the left ventricle of heart 610

In other examples, LCP 604 may monitor or track an LV AV delay period. For example, LCP 604 may monitor an LV AV delay period that begins after each communicated atrial event. LCP 604 may additionally be configured to send a communication signal to LCP 602 directing LCP 602 to deliver a pacing pulse to the left ventricle of heart 610 at the expiration of the LV delay period. In some examples, LCP 604 may wait until the expiration of the LV AV delay period to send a communication signal to LCP 602, and the communication signal may direct LCP 602 to immediately deliver a pacing pulse to the left ventricle of heart 610. In other examples, LCP 604 may send a communication signal to LCP 602 to deliver a pacing pulse to the left ventricle of heart 610 after an amount of time. For example, if the LV AV delay period expires 50 milliseconds from the time LCP 604 sends a communication signal to LCP 602, the communication signal may direct LCP 602 to deliver a pacing pulse to the left ventricle of heart 610 in 50 milliseconds.

In some examples, the LV AV delay period may be shorter or longer than an AV delay period described previously with respect to the right ventricle and tracked by LCP 604 and/or LCP 606. For example, the LV AV delay period may be 100 milliseconds, 50 milliseconds, 25 milliseconds, 10 milliseconds, or any other suitable length of time shorter than the AV delay period. In other examples, the LV AV delay period may be 100 milliseconds, 50 milliseconds, 25 milliseconds, 10 milliseconds, or any other suitable length of time longer than the AV delay period. In still other examples, the LV AV delay period may be substantially equal to the AV delay period. A user may program LCP 604 and/or LCP 602 with an LV AV delay period, for example during a programming session. In some cases, the AV delay period used for the right ventricle and the LV AV delay period used for the left ventricle may be dynamic, and may change depending on the current sensed heart rate of the patient.

In at least some examples, LCP 602 may additionally monitor or track a left ventricular pacing protection interval. LCP 602 may monitor or track the left ventricular pacing protection interval from each sensed left ventricular cardiac event and each paced left ventricular cardiac event. For example, the third IMD may begin a left ventricular pacing protection interval after sensing a left ventricular cardiac event or after delivering a pacing pulse to the left ventricle of heart 610. Such a left ventricular pacing protection interval may be 300 milliseconds, 400 milliseconds, 500 milliseconds, or any other suitable length of time. During a left ventricular pacing protection interval, LCP 602 may be configured to not deliver any pacing pulses to the left ventricle of heart 610. For example, LCP 602 may ignore any expirations of an LV AV delay period that occur during such a left ventricular pacing protection interval. In examples where LCP 602 track an LV AV delay period, LCP 602 may ignore any communication signals from LCP 604 that direct LCP 602 to deliver a pacing pulse to the left ventricle of heart 610 within the left ventricular pacing protection interval.

The above described techniques with three devices are only some examples of how three device systems may operate. In other examples, the devices may be configured to operate according to the techniques disclosed in U.S. Pat. No. 6,438,421, U.S. Pat. No. 6,553,258, U.S. Pat. No. 6,574,506, U.S. Pat. No. 6,829,505 and U.S. Pat. No. 6,871,095, all of which are hereby incorporated by reference herein in their entirety. For example, any of LCPs 602, 604, and/or 606 may be configured to monitor or track other or different intervals and take actions based on those intervals, as described in the references. As with the intervals described herein, any of the devices may monitor or track any of the intervals disclosed in the references and either communicate an expiration of an interval to another device of the system or communicate a direction to take an action based on the expiration of the interval to another device of the system. The additional or different intervals, as disclosed in the references, may provide additional options for operation of multi-device systems implementing multi-chamber therapy.

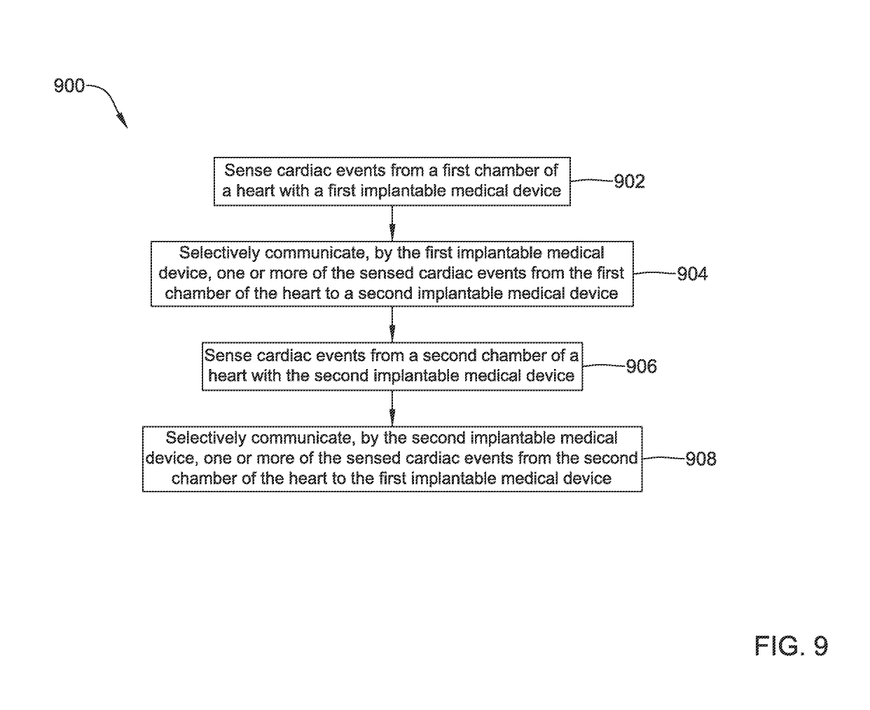

FIG. 9 is a flow diagram of an illustrative method that may be implemented by an implantable medical device system such as shown in any of FIGS. 3-6 including any of the devices described with respect to FIGS. 1 and 2. Although the method of FIG. 9 will be described with respect to the medical device system of FIG. 5, the illustrative method of FIG. 9 may be performed by any suitable medical device system.

In some examples, a first implantable medical device, for instance LCP 506, may be implanted in a first chamber of heart 510, such as an atrium, and may be configured to sense cardiac events from the first chamber of heart 510, as shown at 902. LCP 506 may additionally selectively communicate one or more of the sensed cardiac events from the first chamber of the heart to a second implantable medical device, for example, LCP 502, as shown at 904. LCP 506 may be configured to communicate one or more of the sensed cardiac events using communication signals 714 as described with respect to FIG. 7. A second implantable medical device, for example LCP 502, may be implanted in a second chamber of heart 510, for example a ventricle, and may be configured to sense cardiac events from the second chamber, as shown at 906. LCP 502 may additionally be configured to selectively communicate one or more of the sensed cardiac events from the second chamber of the heart to the first implantable medical device, as shown at 908. For example, LCP 502 may be configured to send communication signals 714 to the first implantable medical device to indicate a sensed cardiac event. LCPs 502 and 506 may additionally selectively communicate the sensed cardiac events in accordance with the techniques described above with respect to FIG. 7.

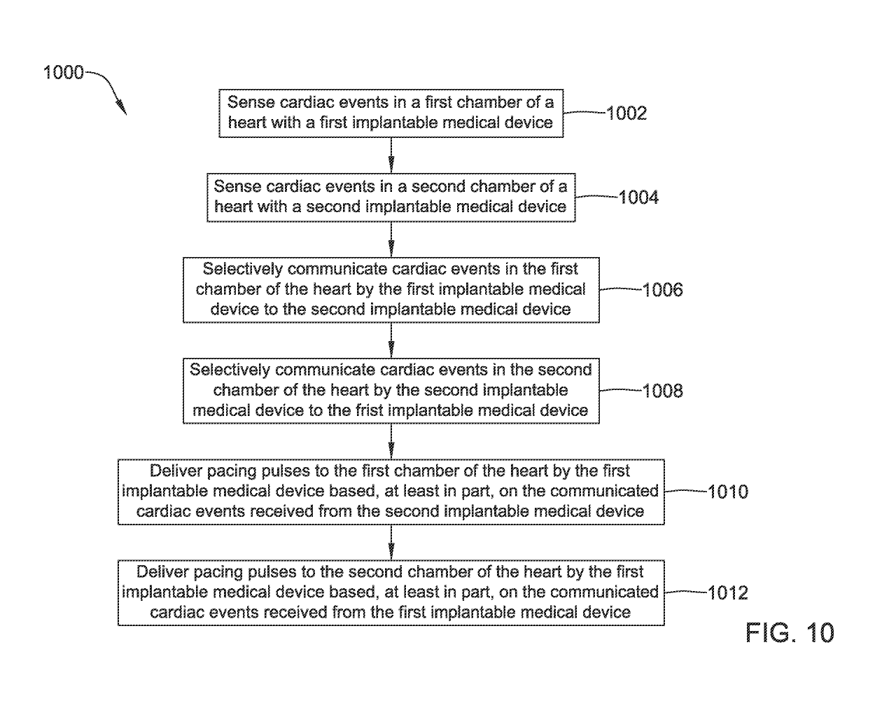

FIG. 10 is a flow diagram of an illustrative method that may be implemented by an implantable medical device system such as shown in any of FIGS. 3-6 including any of the devices described with respect to FIGS. 1 and 2. Although the method of FIG. 10 will be described with respect to the medical device system of FIG. 5, the method of FIG. 10 may be performed by any suitable medical device system.

In some examples, a first implantable medical device, for example LCP 506, may be implanted in a first chamber of heart 510 and configured to sense cardiac events within the first chamber, as shown at 1002. A second implantable medical device, for example LCP 502, may be implanted in a second chamber of heart 510 and configured to sense cardiac events within the second chamber, as shown at 1004. LCP 506 may additionally be configured to selectively communicate cardiac events in the first chamber of heart 510 to the second implantable medical device, as shown at 1006. LCP 506 may be configured to communicate one or more of the sensed cardiac events using communication signals 714 as described with respect to FIG. 7. Additionally, LCP 502 may be configured to selectively communicate cardiac events in the second chamber of the heart to the first implantable medical device, as shown at 1008. For example, LCP 502 may be configured to send communication signals 714 to the first implantable medical device to indicate a sensed cardiac event. LCP 506 may further be configured to deliver pacing pulses to the first chamber of the heart based, at least in part, on the communicated cardiac events received from the second implantable medical device, as shown at 1010. In some examples, the first implantable medical device may track a VA delay period, based at least in part on received communication signals from the second implantable medical device, and deliver a pacing pulse at the expiration of the VA delay period. LCP 502 may further be configured to deliver pacing pulses to the second chamber of the heart based, at least in part, on the communicated cardiac events received from the first implantable medical device, as shown at 1012. In some examples, the second implantable medical device may track an AV delay period, based at least in part on received communication signals from the first implantable medical device, and deliver a pacing pulse at the expiration of the AV delay period.

Those skilled in the art will recognize that the present disclosure may be manifested in a variety of forms other than the specific embodiments described and contemplated herein. As one example, as described herein, various examples include one or more modules described as performing various functions. However, other examples may include additional modules that split the described functions up over more modules than that described herein. Additionally, other examples may consolidate the described functions into fewer modules. Accordingly, departure in form and detail may be made without departing from the scope and spirit of the present disclosure as described in the appended claims.

### Additional Examples

In a first example, a medical system comprises a first leadless cardiac pacemaker (LCP) implantable at a first heart site, a second leadless cardiac pacemaker (LCP) implantable at a second heart site, where the first LCP is configured to communicate information related to a cardiac event that is sensed by the first LCP at the first heart site to the second LCP, and the second LCP is configured to deliver one or more cardiac pacing pulses to one or more pacing electrodes of the second LCP based, at least in part, on the communicated information received from the first LCP.

In a second example, the medical system of the first example may further comprise wherein the second LCP is configured to communicate information related to a cardiac event that is sensed by the second LCP at the second heart site to the first LCP.

In a third example, the medical system of any of the first or second examples may further comprise wherein the first LCP is configured to deliver one or more cardiac pacing pulses to one or more pacing electrodes of the first LCP based, at least in part, on the communicated information received from the second LCP.

In a fourth example, the medical system of any of the first through third examples may further comprise wherein the second LCP is configured to not deliver pacing pulses to the one or more pacing electrodes of the second LCP in the absence of a communicated cardiac event from the first LCP.

In a fifth example, the medical system of any of the first through fourth examples may further comprise, wherein the first LCP is configured to not communicate information related to a sensed cardiac event if the sensed cardiac event is determined to have occurred during a refractory period of the first heart site.

In a sixth example, the medical system of any the first through fifth examples may further comprise wherein the first LCP is configured to not communicate information related to a sensed cardiac event if the sensed cardiac event occurs within a predetermined time of a previous communicated cardiac event.

In a seventh example, the medical system of any of the first through sixth examples may further comprise wherein the first LCP is configured to deliver pacing pulses to the one or more pacing electrodes of the first LCP, and wherein the second LCP is configured to sense the pacing pulses of the first LCP, and the second LCP is configured to deliver the one or more cardiac pacing pulses to one or more pacing electrodes of the second LCP based, at least in part, on the sensed pacing pulses of the first LCP.

In an eighth example, the medical system of any of the first through seventh examples may further comprise wherein the first LCP is configured to sense the pacing pulses of the second LCP, and wherein the first LCP is configured to deliver one or more cardiac pacing pulses to one or more pacing electrodes of the first LCP based, at least in part, on one or more sensed pacing pulses of the second LCP.

In a ninth example, the medical system of any of the first through eighth examples may further comprise wherein the first LCP is configured to communicate information related to the cardiac event that is sensed by the first LCP to the second LCP using one or more communication pulses with an amplitude that is below a capture threshold of the first heart site.

In a tenth example, the medical system of the ninth example may further comprise wherein the one or more communication pulses are bipolar communication pulses.

In an eleventh example, the medical system of any of the first through tenth examples, wherein the first heart site is located in or proximate a first heart chamber and the second heart site is located in or proximate a second heart chamber.

In a twelfth example, a method of communicating cardiac events between a plurality of implantable medical devices may comprise sensing cardiac events from a first chamber of a heart with a first implantable medical device, selectively communicating, by the first implantable medical device, one or more of the sensed cardiac events from the first chamber of the heart to a second implantable medical device, sensing cardiac events from a second chamber of a heart with the second implantable medical device, and selectively communicating, by the second implantable medical device, one or more of the sensed cardiac events from the second chamber of the heart to the first implantable medical device.

A thirteenth example may comprise the method of the twelfth example wherein selectively communicating, by the first implantable medical device, one or more of the sensed cardiac events from the first chamber of the heart to the second implantable medical device comprises not communicating sensed cardiac events that occur within a predefined post ventricular atrial refractory time period (PVARP).

A fourteenth example may comprise the method of any of the twelfth and thirteenth examples wherein selectively communicating, by the first implantable medical device, one or more of the sensed cardiac events from the first chamber of the heart to the second implantable medical device comprises not communicating sensed cardiac events that occur before expiration of a blocking period following a last communication of a sensed cardiac event by the first implantable medical device.

In a fifteenth example, the method of any of the twelfth through fourteenth examples may further comprise, delivering, by the second implantable medical device, a pacing pulse to the second chamber of the heart following a predefined atrioventricular (AV) delay period in response to receiving the sensed cardiac event from the first implantable medical device.

A sixteenth example may comprise the method of the fifteenth example wherein delivering, by the second implantable medical device, a pacing pulse to the second chamber of the heart following the predefined AV delay period in response to receiving the sensed cardiac event from the first implantable medical device comprises delivering, by the second implantable medical device, a pacing pulse to the second chamber of the heart after the predefined AV delay time period in response to receiving a sensed cardiac event from the first implantable medical device unless the second implantable medical device senses a cardiac event from the second chamber of the heart within the predefined AV delay period.

In a seventeenth example, the method of any of the twelfth through sixteenth examples may further comprise delivering, by the second implantable medical device, a pacing pulse after a predefined lower rate limit interval (LRLI) following a previous sensed cardiac event from the second chamber of the heart or a previous pacing pulse delivered to the second chamber of the heart.

An eighteenth example may comprise the method of any of the twelfth through seventeenth examples wherein communicating comprises delivering a conducted communication pulse.

A nineteenth example may comprise the method of any of the twelfth through eighteenth examples wherein the first implantable medical device is implanted in or proximate an atrium of the heart and the second implantable medical device is implanted in or proximate a ventricle of the heart.

In a twentieth example, a method for delivering CRT therapy to a heart of a patient comprises sensing cardiac events in a first chamber of the heart with a first implantable medical device, sensing cardiac events in a second chamber of the heart with a second implantable medical device, selectively communicating cardiac events in the first chamber of the heart by the first implantable medical device to the second implantable medical device, selectively communicating cardiac events in the second chamber of the heart by the second implantable medical device to the first implantable medical device, delivering pacing pulses to the first chamber of the heart by the first implantable medical device based, at least in part, on the communicated cardiac events received from the second implantable medical device, and delivering pacing pulses to the second chamber of the heart by the first implantable medical device based, at least in part, on the communicated cardiac events received from the first implantable medical device.

In a twenty-first example, a medical system comprises a first leadless cardiac pacemaker (LCP) implantable at a first heart site, a second leadless cardiac pacemaker (LCP) implantable at a second heart site, the first LCP is configured to communicate information related to a cardiac event that is sensed by the first LCP at the first heart site to the second LCP, and the second LCP is configured to deliver one or more cardiac pacing pulses to one or more pacing electrodes of the second LCP based, at least in part, on the communicated information received from the first LCP.

In a twenty-second example, the medical system of the twenty-first example further comprises wherein the second LCP is configured to communicate information related to a cardiac event that is sensed by the second LCP at the second heart site to the first LCP.

In a twenty-third example, the medical system of any of the twenty-first and twenty-second examples further comprises wherein the first LCP is configured to deliver one or more cardiac pacing pulses to one or more pacing electrodes of the first LCP based, at least in part, on the communicated information received from the second LCP.

In a twenty-fourth example, the medical system of any of the twenty-first through twenty-third examples further comprises wherein the second LCP is configured to not deliver pacing pulses to the one or more pacing electrodes of the second LCP in the absence of a communicated cardiac event from the first LCP.

In a twenty-fifth example, the medical system of any of the twenty-first, twenty-third, and twenty-fourth examples further comprises wherein the first LCP is configured to not communicate information related to a sensed cardiac event if the sensed cardiac event is determined to have occurred during a refractory period of the first heart site.

In a twenty-sixth example, the medical system of any of the twenty-first through twenty-fifth examples further comprises wherein the first LCP is configured to not communicate information related to a sensed cardiac event if the sensed cardiac event occurs within a predetermined time of a previous communicated cardiac event.

In a twenty-seventh example, the medical system of any of the twenty-first through twenty-sixth examples further comprises wherein the first LCP is configured to deliver pacing pulses to the one or more pacing electrodes of the first LCP, and wherein the second LCP is configured to sense the pacing pulses of the first LCP, and the second LCP is configured to deliver the one or more cardiac pacing pulses to one or more pacing electrodes of the second LCP based, at least in part, on the sensed pacing pulses of the first LCP.

In a twenty-eighth example, the medical system of claim of any of the twenty-first through twenty-seventh examples further wherein the first LCP is configured to sense the pacing pulses of the second LCP, and wherein the first LCP is configured to deliver one or more cardiac pacing pulses to one or more pacing electrodes of the first LCP based, at least in part, on one or more sensed pacing pulses of the second LCP.

In a twenty-ninth example, the medical system of any of the twenty-first through twenty-eighth examples further comprises wherein the first LCP is configured to communicate information related to the cardiac event that is sensed by the first LCP to the second LCP using one or more communication pulses with an amplitude that is below a capture threshold of the first heart site.

In thirtieth example, the medical system of the twenty-ninth example further comprises wherein the one or more communication pulses are bipolar communication pulses.

In thirty-first example, the medical system of claim of any of the twenty-first through thirtieth examples further comprises wherein the first heart site is located in or proximate an atrium of the heart.

In a thirty-second example, the medical system of any of the twenty-first through thirty-first examples further comprises wherein the second heart site is located in or proximate a ventricle of the heart.

In a thirty-third example, the medical system of any of the twenty-first through thirty-second examples further comprises wherein the second LCP is further configured to deliver a pacing pulse to the second heart site following a predefined atrioventricular (AV) delay period in response to receiving the sensed cardiac event from the first implantable medical device.

In a thirty-fourth example, the medical system of claim of any of the twenty-first through thirty-third examples further wherein the second LCP is further configured to deliver a pacing pulse after a predefined lower rate limit interval (LRLI) following a previous sensed cardiac event at the second heart site or a previous pacing pulse delivered to the second heart site.

In a thirty-fifth example, the medical system of any of the twenty-first through thirty-fourth examples further comprises wherein the first LCP is further configured to only communicate information related to a cardiac event that is sensed by the first LCP at the first heart site to the second LCP if the cardiac event occurs outside of a predefined post ventricular atrial refractory time period (PVARP).

What is claimed is:

1. A medical system comprising:
a first leadless cardiac pacemaker (LCP) implantable at a first heart site;
a second leadless cardiac pacemaker (LCP) implantable at a second heart site;
the first LCP is configured to communicate information related to a cardiac event that is sensed by the first LCP at the first heart site to the second LCP; and
the second LCP is configured to deliver one or more cardiac pacing pulses to one or more pacing electrodes of the second LCP based, at least in part, on the communicated information received from the first LCP.

2. The medical system of claim 1, wherein the second LCP is configured to communicate information related to a cardiac event that is sensed by the second LCP at the second heart site to the first LCP.

3. The medical system of claim 2, wherein the first LCP is configured to deliver one or more cardiac pacing pulses to one or more pacing electrodes of the first LCP based, at least in part, on the communicated information received from the second LCP.

4. The medical system of claim 1, wherein the second LCP is configured to not deliver pacing pulses to the one or more pacing electrodes of the second LCP in the absence of a communicated cardiac event from the first LCP.

5. The medical system of claim 1, wherein the first LCP is configured to not communicate information related to a sensed cardiac event if the sensed cardiac event is determined to have occurred during a refractory period of the first heart site.

6. The medical system of claim 1, wherein the first LCP is configured to not communicate information related to a sensed cardiac event if the sensed cardiac event occurs within a predetermined time of a previous communicated cardiac event.

7. The medical system of claim 1, wherein the first LCP is configured to deliver pacing pulses to the one or more pacing electrodes of the first LCP, and wherein the second LCP is configured to sense the pacing pulses of the first LCP, and the second LCP is configured to deliver the one or more cardiac pacing pulses to one or more pacing electrodes of the second LCP based, at least in part, on the sensed pacing pulses of the first LCP.

8. The medical system of claim 1, wherein the first LCP is configured to sense the pacing pulses of the second LCP, and wherein the first LCP is configured to deliver one or more cardiac pacing pulses to one or more pacing electrodes of the first LCP based, at least in part, on one or more sensed pacing pulses of the second LCP.

9. The medical system of claim 1, wherein the first LCP is configured to communicate information related to the cardiac event that is sensed by the first LCP to the second LCP using one or more communication pulses with an amplitude that is below a capture threshold of the first site.

10. The medical system of claim 9, wherein the one or more communication pulses are bipolar communication pulses.

11. The medical system of claim 1, wherein the first heart site is located in or proximate a first heart chamber and the second heart site is located in or proximate a second heart chamber.

12. A method of communicating cardiac events between a plurality of implantable medical devices, the method comprising:
sensing cardiac events from a first chamber of a heart with a first implantable medical device;
selectively communicating, by the first implantable medical device, one or more of the sensed cardiac events from the first chamber of the heart to a second implantable medical device;
sensing cardiac events from a second chamber of a heart with the second implantable medical device; and
selectively communicating, by the second implantable medical device, one or more of the sensed cardiac events from the second chamber of the heart to the first implantable medical device.

13. The method of claim 12, wherein selectively communicating, by the first implantable medical device, one or more of the sensed cardiac events from the first chamber of the heart to the second implantable medical device comprises not communicating sensed cardiac events that occur within a predefined post ventricular atrial refractory time period (PVARP).

14. The method of claim 12, wherein selectively communicating, by the first implantable medical device, one or more of the sensed cardiac events from the first chamber of the heart to the second implantable medical device comprises not communicating sensed cardiac events that occur before expiration of a blocking period following a last communication of a sensed cardiac event by the first implantable medical device.

15. The method of claim 12, further comprising, delivering, by the second implantable medical device, a pacing pulse to the second chamber of the heart following a predefined atrioventricular (AV) delay period in response to receiving the sensed cardiac event from the first implantable medical device.

16. The method of claim 15, wherein delivering, by the second implantable medical device, a pacing pulse to the second chamber of the heart following the predefined AV delay period in response to receiving the sensed cardiac event from the first implantable medical device comprises delivering, by the second implantable medical device, a pacing pulse to the second chamber of the heart after the predefined AV delay time period in response to receiving a sensed cardiac event from the first implantable medical device unless the second implantable medical device senses a cardiac event from the second chamber of the heart within the predefined AV delay period.

17. The method of claim 12, further comprising delivering, by the second implantable medical device, a pacing pulse after a predefined lower rate limit interval (LRLI) following a previous sensed cardiac event from the second chamber of the heart or a previous pacing pulse delivered to the second chamber of the heart.

18. The method of claim 12, wherein communicating comprises delivering a conducted communication pulse.

19. The method of claim 12, wherein the first implantable medical device is implanted in or proximate an atrium of the heart and the second implantable medical device is implanted in or proximate a ventricle of the heart.

20. A method for delivering CRT therapy to a heart of a patient, the method comprising:
sensing cardiac events in a first chamber of the heart with a first implantable medical device;
sensing cardiac events in a second chamber of the heart with a second implantable medical device;
selectively communicating cardiac events in the first chamber of the heart by the first implantable medical device to the second implantable medical device;
selectively communicating cardiac events in the second chamber of the heart by the second implantable medical device to the first implantable medical device;
delivering pacing pulses to the first chamber of the heart by the first implantable medical device based, at least in part, on the communicated cardiac events received from the second implantable medical device; and
delivering pacing pulses to the second chamber of the heart by the first implantable medical device based, at least in part, on the communicated cardiac events received from the first implantable medical device.
